(19) United States
(12) Patent Application Publication
(10) Pub. No.: US 2026/0053793 A1
(43) Pub. Date: Feb. 26, 2026

(54) USE OF BERBERINE DERIVATIVE NBD-125 IN PREPARATION OF DRUG FOR PREVENTING AND/OR TREATING OBESITY

(71) Applicant: Xiamen Danning Traditional Chinese Medicine Research Institute Co., Ltd., Xiamen (CN)

(72) Inventors: Tianhui HU, Xiamen (CN); Wenqing ZHANG, Xiamen (CN); Shaoliang ZHANG, Xiamen (CN); Yanhong FENG, Xiamen (CN); Beibei XU, Xiamen (CN); Yandong ZHANG, Xiamen (CN); Xiaoting HONG, Xiamen (CN); Sijie CHENG, Xiamen (CN)

(21) Appl. No.: 18/979,097

(22) Filed: Dec. 12, 2024

(30) Foreign Application Priority Data
Aug. 26, 2024 (CN) 202411170945.8

(51) Int. Cl. A61K 31/4745; A61P 3/04

(52) U.S. Cl. CPC A61K 31/4745; A61P 3/04

(57) ABSTRACT

The use of a berberine derivative NBD-125 in preparation of a drug for preventing and/or treating obesity is provided, belonging to the technical field of obesity treatment. Use of a berberine derivative NBD-125 in preparation of a drug for preventing and/or treating obesity is provided. The berberine derivative NBD-125 can prevent and/or treat obesity, with an effect significantly better than that of berberine. Moreover, the berberine derivative NBD-125 can inhibit weight gain, enhance metabolism and/or heat production, inhibit white adipose production, and inhibit differentiation of a preadipocyte into an adipocyte and/or lipid accumulation, where each inhibition index is better than that of the berberine.

### CROSS-REFERENCE TO RELATED APPLICATION

This patent application claims the benefit and priority of Chinese Patent Application No. 202411170945.8 filed with the China National Intellectual Property Administration on Aug. 26, 2024, the disclosure of which is incorporated by reference herein in its entirety as part of the present application.

### TECHNICAL FIELD

The present disclosure belongs to the technical field of obesity treatment, and specifically relates to the use of a berberine derivative NBD-125 in preparation of a drug for prevention and/or treatment of obesity.

### BACKGROUND

With the significant changes in people's quality of life and lifestyle, the incidence of obesity has been increasing and its growth trend has become more obvious. Overweight and obesity are prevalent around the world and have become one of the serious public health issues.

Obesity refers to an imbalance in energy homeostasis, excessive accumulation and/or abnormal distribution of body adipose, and weight gain due to excessive food intake or changes in body metabolism, and is a chronic metabolic disease caused by the interaction of multiple factors. Studies have found that obese people are more likely to suffer from diseases such as hyperlipidemia and hypertension^{[1-3]}. In addition to lifestyle changes, diet control, exercise, medication, and surgery, a variety of drugs have been tried clinically to treat obesity (such as thyroid extract, 2-nitrophenol, and amphetamine), but their efficacy and safety are not ideal. Therefore, developing novel drugs to treat obesity and exploring effective intervention methods for obesity are issues that need to be addressed urgently.

Berberine (BBR for short), an active ingredient monomer of traditional Chinese medicines such as Rhizoma Coptidis and Cortex Phellodendri, has been used to treat dysentery and enteritis. In recent years, studies have found that berberine can also inhibit tumor growth, lower blood glucose, and reduce blood lipids, showing a potential weight loss effect^{[4-6]} However, the extremely low solubility and bioavailability of berberine greatly limit its clinical application. In our previous study, a berberine derivative NBD-125 was developed with significantly improved solubility and bioavailability while retaining the safety and low toxicity of berberine^{[7]}. However, the potential of the berberine derivative NBD-125 in the treatment of obesity has not been studied.
[1] ROSEN E D, SPIEGELMAN B M. What we talk about when we talk about fat [J]. Cell, 2014, 156 (1-2): 20-44.
[2] YANOVSKI J A. Obesity: Trends in underweight and obesity—scale of the problem [J]. Nat Rev Endocrinol, 2018, 14 (1): 5-6.
[3] BLUHER M. Obesity: global epidemiology and pathogenesis [J]. Nature Reviews Endocrinology, 2019, 15 (5): 288-98.
[4] Leng S H, Lu F R, Xu L J. Therapeutic effects of berberine in impaired glucose tolerance rats and its influence on insulin secretion. Acta Pharm. Sin. 2004, 25:496-502.
[5] Lee Y S, Kim W S, Kim K H, Yoon M J, Cho H J, Shen Y, et al. Berberine, a natural plant product, activates AMP-activated protein kinase with beneficial metabolic effects in diabetic and insulin-resistant states. Diabetes 2006, 55: 2256-64.
[6] Yin J, Gao Z G, Liu D, Liu Z J, Ye J P. Berberine improves glucose metabolism through induction of glycolysis. Am J Physiol Endocrinol Metab 2008, 294: E148-56.
[7] Xu B, Jiang X, Xiong J, Lan J, Zhong L, Wang X, Cao H, Zhan Y, Zhang Y, Hu T. Structure-Activity Relationship Study Enables the Discovery of a Novel Berberine Analogue as RXRα Activator to Inhibit Colon Cancer. J. Med. Chem. 2020, 63: 5841-5855.

### SUMMARY

An objective of the present disclosure is to provide the use of a berberine derivative NBD-125 in preparation of a drug for preventing and/or treating obesity. The berberine derivative NBD-125 can achieve the prevention and/or treatment of obesity.

Use of a berberine derivative NBD-125 in preparation of a drug for preventing and/or treating obesity is provided.

Preferably, the obesity includes high-fat diet (HFD)-induced obesity.

The present disclosure further provides the use of a berberine derivative NBD-125 in preparation of a drug for inhibiting weight gain.

The present disclosure further provides the use of a berberine derivative NBD-125 in preparation of a drug for enhancing metabolism and/or heat production.

The present disclosure further provides the use of a berberine derivative NBD-125 in preparation of a drug for inhibiting white adipose production.

Preferably, the inhibition of white adipose production includes inhibiting white adipose gain and/or inhibiting white adipocyte enlargement.

The present disclosure further provides the use of a berberine derivative NBD-125 in preparation of a drug for enhancing brown adipocyte function.

The present disclosure further provides the use of a berberine derivative NBD-125 in preparation of a drug for inhibiting the differentiation of a preadipocyte into an adipocyte and/or lipid accumulation.

Preferably, the inhibition of the differentiation of the preadipocyte into the adipocyte includes inhibiting mitotic cloning of the preadipocyte at an early stage of the differentiation.

Preferably, the structure of the berberine derivative NBD-125 is shown in formula I.

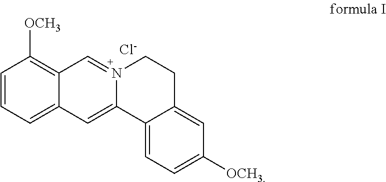

The use of a berberine derivative NBD-125 in preparation of a drug for preventing and/or treating obesity is provided. The berberine derivative NBD-125 can prevent and/or treat obesity, with a significantly better effect than that of berberine. Moreover, the berberine derivative NBD-125 can inhibit weight gain, enhance metabolism and/or heat production, inhibit white adipose production, and inhibit differentiation of a preadipocyte into an adipocyte and/or lipid accumulation, where each inhibition index is better than that of berberine.

### BRIEF DESCRIPTION OF THE DRAWINGS

To illustrate the examples of the present disclosure or the technical solutions in the prior art more clearly, the accompanying drawings required in the examples will be briefly introduced below. In the drawings, descriptions, and examples, NBD-125 is abbreviated as B125, and berberine is represented by its English abbreviation BBR. Apparently, the accompanying drawings in the following description show merely some embodiments of the present disclosure, and those of ordinary skill in the art may still derive other drawings from these accompanying drawings without creative efforts.

FIG. 1A-FIG. 1H show the weight loss effect of B125 provided by the present disclosure in C57BL/6 mice induced by a HFD; where FIG. 1A is the weight change of mice in the HFD group during B125 administration; FIG. 1B is the total amount of white adipose tissue (WAT) of each group of mice; FIG. 1C is the weight change of subcutaneous adipose tissue of each group of mice; FIG. 1D is the weight change of visceral adipose tissue of each group of mice; FIG. 1E is the weight change of brown adipose tissue (BAT) of each group of mice; FIG. 1F is a representative image of hematoxylin-eosin (H&E) staining of white adipose and brown adipose of each group of mice; FIG. 1G is the statistical graph of the proportion of white adipocytes of different sizes in mice in the HFD group; FIG. 1H is the statistical graph of the proportion of brown adipocytes of different sizes in mice in the HFD group; n=6, * compared with HFD-Saline, *P<0.05, **P<0.01, ***P<0.005, ****P<0.001; # compared with HFD-BBR, #P<0.05, ##P<0.01, ###P<0.005, ####P<0.001;

FIG. 2A-FIG. 2D show the results of B125 provided by the present disclosure in enhancing the metabolism and heat production of HFD-induced obesity C57BL/6 mice; where FIG. 2A is the statistical graph of O₂ consumption in the light cycle (7 to 19 o'clock) and dark cycle (19 to 7 o'clock) of HFD mice; FIG. 2B is the statistical graph of CO₂ production in the light cycle and dark cycle of HFD mice; FIG. 2C is the statistical graph of heat release in the light cycle and dark cycle of HFD mice; FIG. 2D is the statistical graph of the respiratory exchange rate (RER) in the light cycle and dark cycle of HFD mice; (n=6, * compared with HFD-Saline, *P<0.05; #HFD-B125 compared with HFD-BBR, #P<0.05);

FIG. 3A-FIG. 3L show the results of B125 provided by the present disclosure in inhibiting mitotic cloning of preadipocytes; where FIG. 3A-FIG. 3K are the cell cycle distribution diagram of the early mitotic cloning stage of preadipocyte differentiation detected by flow cytometry after B125 treatment for different time periods; un: 0 h of induction of differentiation and drug treatment (FIG. 3A); 16h-Vehicle: induced differentiation 16h+B125 solvent DMSO treatment group (FIG. 3B); 16h-B-5 μM: induced differentiation 16 h+5 μM BBR treatment 16 h group (FIG. 3C); 16h-B-10 M: induced differentiation 16 h+10 μM BBR treatment 16 h group (FIG. 3D); 16h-B125-5 M: induced differentiation 16 h+5 M B125 treatment 16 h group (FIG. 3E); 16h-B125-10 M: induced differentiation 16 h+10 μM B125 treatment 16 h group (FIG. 3F); 28h-Vehicle: induced differentiation 28 h+B125 solvent DMSO treatment group (FIG. 3G); 28h-B-5 m: induced differentiation 28 h+5 μM BBR treatment 26 h group (FIG. 3H); 28h-B-10 μM: induced differentiation 28 h+10 M BBR treatment 28 h group (FIG. 3I); 28h-B125-5 μM: induced differentiation 28 h+5 M B125 treatment 28 h group (FIG. 3J); 28h-B125-10 μM: induced differentiation 28 h+10 μM B125 treatment 28 h group (FIG. 3K); FIG. 3L is the period distribution statistical diagram; left: 16 h; right: 28 h;

FIG. 4A-FIG. 4B show the results of B125 provided by the present disclosure in inhibiting the differentiation of preadipocytes and intracellular lipid accumulation; where FIG. 4A is a representative image of oil red O staining (bottom) and phase contrast image (top); FIG. 4B is the lipid accumulation content of 3T3-L1 adipocytes; n=3, * compared with HFD-Vehicle, *P<0.05, **P<0.01, ***P<0.005, ****P<0.001; #HFD-B125 compared with HFD-BBR, #P<0.05.

### DETAILED DESCRIPTION OF THE EMBODIMENTS

The present disclosure provides the use of a berberine derivative NBD-125 (NBD-125, abbreviated as B125) in preparation of a drug for preventing and/or treating obesity. In the present disclosure, obesity preferably includes TED-induced obesity. There is no special limitation on the source of the berberine derivative NBD-125, which can be prepared by conventional methods. For example, reference may be made to the patent application “CN201910484491.4”, namely “A berberine derivative NBD-125 and its application”. The berberine derivative NBD-125 has a structure shown in formula I.

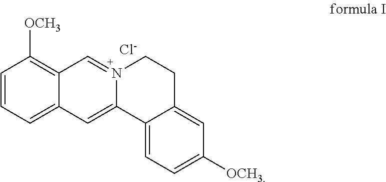

The present disclosure further provides the use of a berberine derivative NBD-125 in preparation of a drug for inhibiting weight gain. In the present disclosure, the weight gain is preferably TED-induced weight gain. Test results show that the berberine derivative NBD-125 can significantly inhibit the weight gain of TED-induced C57BL/6 obesity mice, and has no significant effect on their food intake.

The present disclosure further provides the use of a berberine derivative NBD-125 in preparation of a drug for enhancing metabolism and/or heat production. In the present disclosure, the enhancing metabolism and/or heat production is preferably enhancement of HFD-induced metabolism and/or heat production. Test results show that the berberine derivative NBD-125 significantly increases the oxygen consumption, carbon dioxide production, and heat production of HFD-induced mice, but does not change their RER, indicating that B125 can significantly enhance the metabolism and heat production of TED-induced obesity mice, thereby inhibiting their weight gain.

The present disclosure further provides the use of a berberine derivative NBD-125 in preparation of a drug for inhibiting white adipose production.

In the present disclosure, the inhibiting white adipose production includes inhibiting white adipose gain and/or inhibiting white adipocyte enlargement. The NBD-125 can significantly inhibit the increase of white adipose in the TED-induced C57BL/6 obesity mice; the NBD-125 can also significantly inhibit the enlargement of white adipocytes, exerting the effect of inhibiting the white adipose production.

The present disclosure further provides the use of a berberine derivative NBD-125 in preparation of a drug for enhancing brown adipocyte function. In the present disclosure, the enhancement of a brown adipocyte function preferably includes enhancing the metabolic and heat production functions of BAT. The enhancement of the function of the fatty acid cells preferably includes increasing the number of small brown adipocytes without changing the weight of the BAT. The main function of brown adipocytes is heat production. The greater the relative proportion of BAT, the higher the metabolic rate and the less likely to become obese. While the weight of BAT remains unchanged, smaller cells (less adipose content) indicate stronger weight loss function and potential. The experimental results show that under the treatment of NBD-125, the weight of brown adipose remains unchanged, but the number of cells with an area of (200-500) μm² in the BAT is significantly reduced, while the number of cells with an area of (10-50) μm² is significantly increased, indicating enhanced metabolic function and greater weight loss potential.

The present disclosure further provides the use of a berberine derivative NBD-125 in preparation of a drug for inhibiting differentiation of a preadipocyte into an adipocyte and/or lipid accumulation. In the present disclosure, the inhibition of differentiation of the preadipocyte into the adipocyte includes inhibiting mitotic cloning of the preadipocyte at the early stage of the differentiation. Test results show that NBD-125 can significantly block the mitotic cloning in an early stage of 3T3-L1 differentiation, thereby inhibiting the entire process of adipogenic differentiation; NBD-125 can also significantly inhibit the lipid accumulation of 3T3-L1 preadipogenic differentiation.

In order to further illustrate the present disclosure, the use of a berberine derivative NBD-125 in preparation of a drug for preventing and/or treating obesity provided by the present disclosure is described in detail below with reference to the accompanying drawings and examples, but the accompanying drawings and the examples should not be construed as limiting the protection scope of the present disclosure.

### Example 1

### I. In Vivo Animal Model

Four-week-old male C57BL/6J mice (SPF grade) were purchased from the Experimental Animal Center of Xiamen University and raised in a room at 24° C. to 26° C. with humidity control, given free access to food and sterile water; the mice were used for experiments after acclimating to the environment for 2 weeks. For specific diet studies, 6-week-old C57BL/6J mice were randomized into normal fat diet and HFD groups. The normal fat diet group (NFD) still maintained a standard diet, while the HFD group was given a HFD (60% adipose, Nantong Trophic Animal Feed High-Tech Co., Ltd, China; Cat. No. TP23400) for 8 weeks. The HFD group was then randomly divided into three groups and intraperitoneally injected with normal saline (0.9% Saline), 10 μM/kg BBR (positive control), and 10 μM/kg B125 every other day for 60 d. The three groups were injected with equal volumes, and the molar/body weight concentrations of BBR and B125 were equal.

### 1. Influence of B125 on Body Weight of HFD-Induced Obesity C57BL/6 Mice

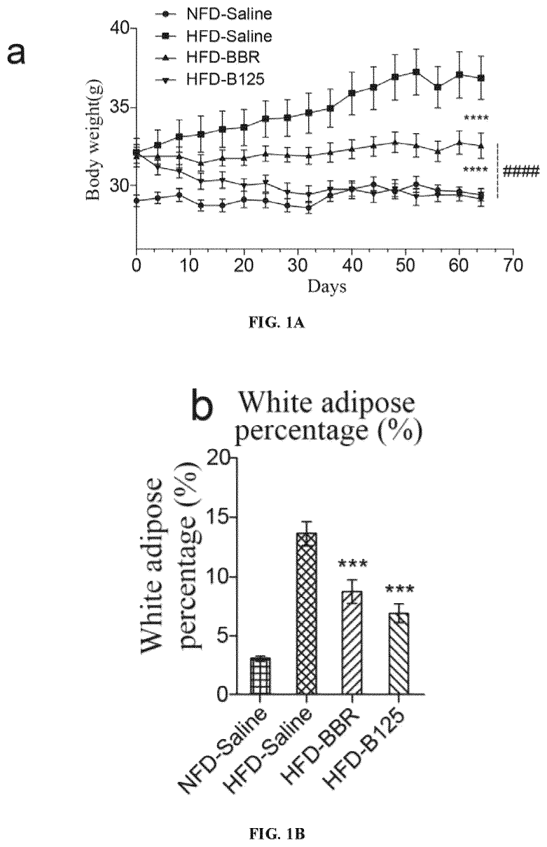

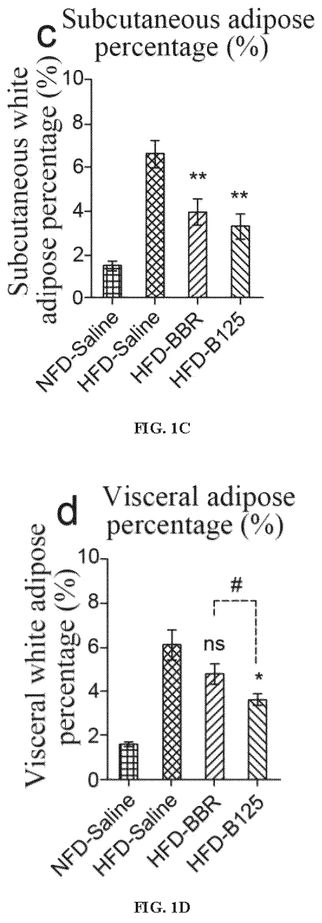

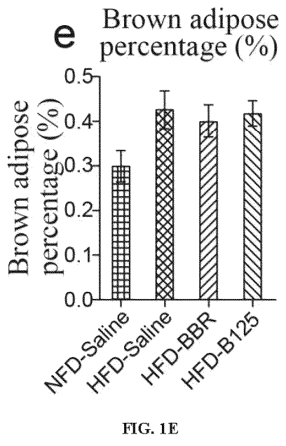

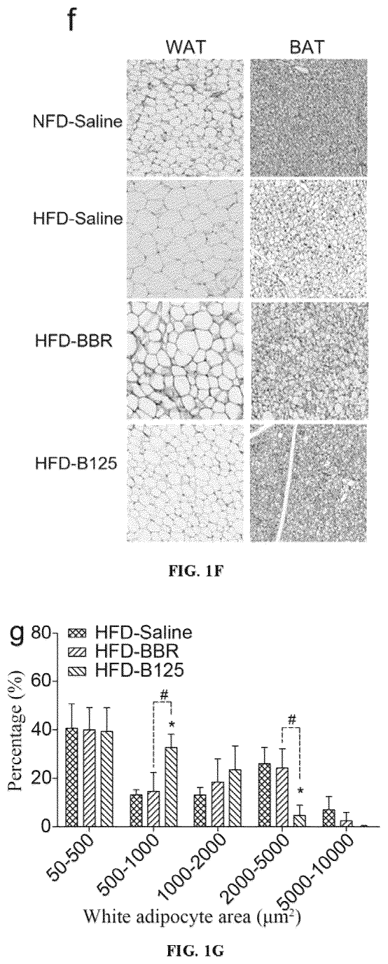

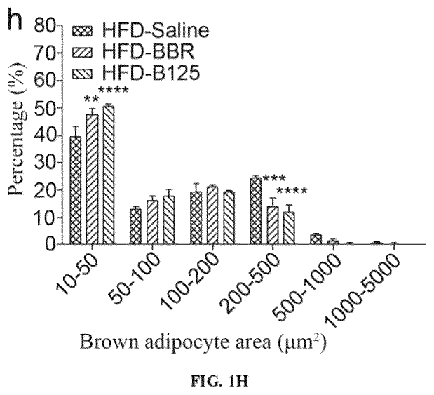

During the above experiments, the body weight and food intake of mice were measured regularly (every 4 days). On day 0, the average body weight of mice in the HFD group was similar (HFD-Saline group: 32.13 g; HFD-BBR group: 31.87 g; HFD-B125 group: 32.01 g). Compared with the HFD-Saline group, the average body weight of mice in the HFD-B125 group was significantly reduced, with significant differences of p<0.05 on day 8 and p<0.001 on day 20. Compared with the HFD-BBR group, the average body weight of mice in the HFD-B125 group gradually decreased, with significant differences of p<0.05 on day 28 and p<0.001 on day 52. At the same time, the average body weight of mice in the HFD-BBR group was significantly lower than that in the HFD-Saline group on day 20 (p<0.05) and p<0.001 on day 56. Therefore, B125 treatment could significantly inhibit the weight gain of HFD-induced C57BL/6 obesity mice (FIG. 1A), with an effect significantly superior to that of BBR. The changes in food intake of each group of mice during the experiment were observed and statistically analyzed, and it was found that the drug had no significant effect on food intake.

### 2. Influence of B125 on Metabolism and Heat Production in HFD-Induced Obesity C57BL/6 Mice

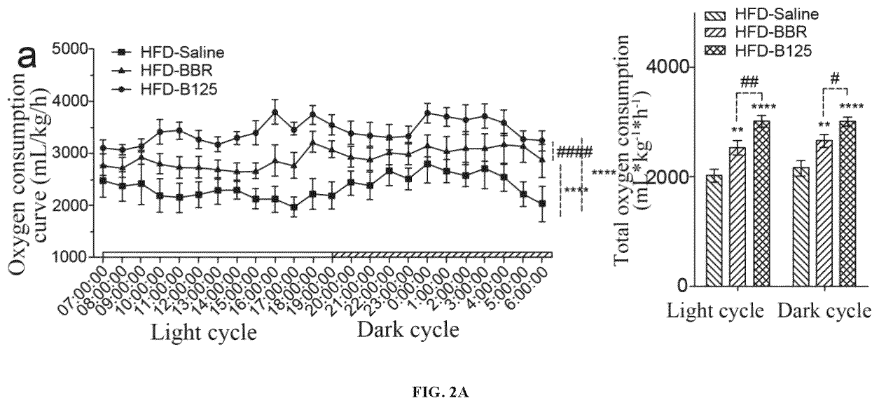

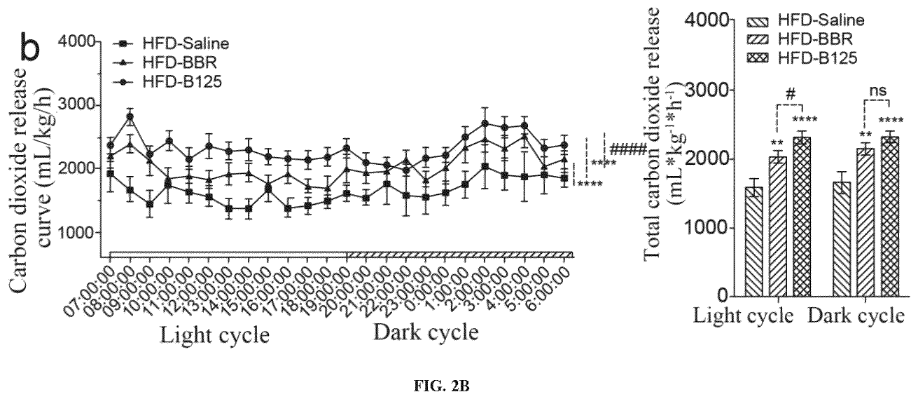

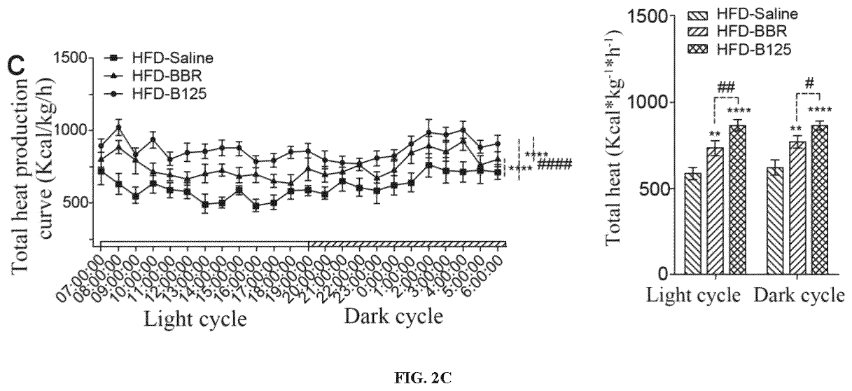

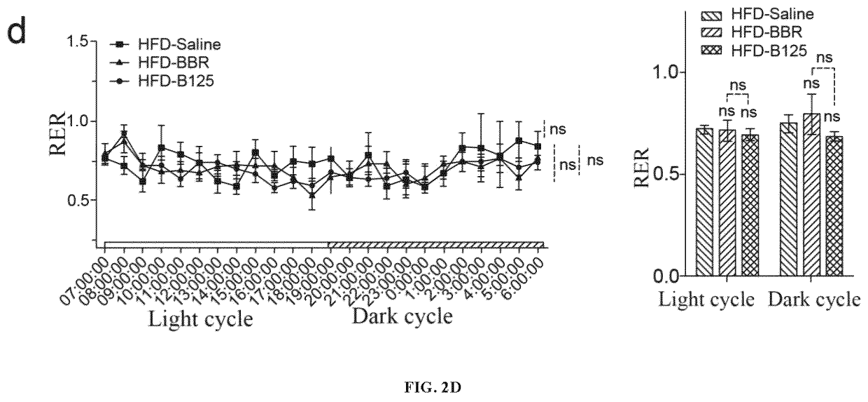

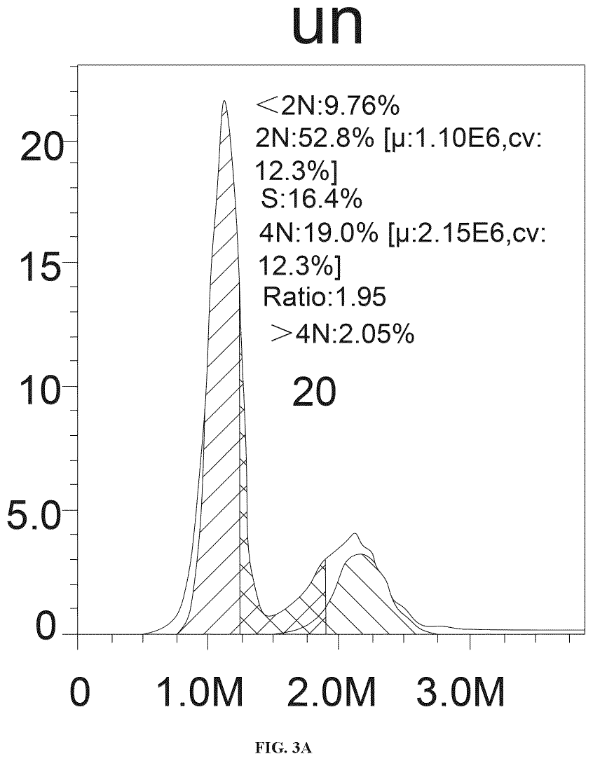

After 30 intraperitoneal injections (60 d), mice in different treatment groups of HFD (HFD-Saline, HFD-BBR, and HFD-B125) were placed in a Sable Promethion calorimetric system (metabolic cage), 1 mouse/cage. The instrument automatically recorded the oxygen consumption, carbon dioxide release, heat production, RER, food intake, as well as the X-axis, Y-axis, and Z-axis displacements of mice in the metabolic cage (representing their exercise capacity) every five minutes for more than 3 days (72 h). The middle 24-h data were selected for statistical analysis, where 7:00 to 19:00 was marked as a light cycle (Light Cycle), and 19:00 to 7:00 was marked as a dark cycle (Dark Cycle). The oxygen consumption, carbon dioxide production, heat production, RER, and other indicators of each mouse in each group (n=5) were calculated by dividing each data by the weight of the mouse, and their average values were plotted (FIG. 2 for details of some indicators) and compared between groups. The test results showed that B125 did not change the food intake of mice, but significantly increased the oxygen consumption (FIG. 2A), carbon dioxide production (FIG. 2B), and heat production (FIG. 2C) of mice fed with HFD, but did not change the RER (FIG. 2D). Specifically, as shown by the oxygen consumption curve on the left side of FIG. 2A, the average oxygen consumption (mL/kg/h) of mice in the HFD-B125 group was significantly higher than that of the HFD-Saline and HFD-BBR groups. The light cycle and dark cycle statistics on the right side showed that: during the light cycle, the average oxygen consumption of the three groups of mice was 2,028.33 mL/kg/h, 2,539.70 mL/kg/h, and 3,022.68 mL/kg/h, respectively. The statistical results showed that HFD-B125 vs HFD-Saline: p<0.001, HFD-BBR vs HFD-Saline: p<0.01, HFD-B125 vs HFD-BBR: p<0.01. During the dark cycle, the average oxygen consumption of the three groups of mice was 2,158.30 mL/kg/h, 2,663.98 mL/kg/h, and 3,014.18 mL/kg/h, respectively. The statistical results showed that HFD-B125 vs HFD-Saline: p<0.001, HFD-BBR vs HFD-Saline: p<0.01, HFD-B125 vs HFD-BBR: p<0.05. Therefore, the effect of B125 was significantly better than that of BBR in both light and dark cycles. As shown in the carbon dioxide release curve on the left side of FIG. 2B, the average carbon dioxide release (mL/kg/h) of mice in the HFD-B125 group was significantly higher than that in the HFD-Saline and HFD-BBR groups. The light cycle and dark cycle statistics on the right side showed that: during the light cycle, the carbon dioxide consumption of the three groups of mice was 1,569.16 mL/kg/h, 2,017.23 mL/kg/h, and 2,260.81 mL/kg/h, respectively. The statistical results showed that HFD-B125 vs HFD-Saline: p<0.001, HFD-BBR vs HFD-Saline: p<0.01, HFD-B125 vs HFD-BBR: p<0.05. During the dark cycle, the carbon dioxide consumption of the three groups of mice was 1,645.49 mL/kg/h, 2,140.42 mL/kg/h, and 2,281.00 mL/kg/h, respectively. The statistical results showed that HFD-B125 vs HFD-Saline: p<0.001, HFD-BBR vs HFD-Saline: p<0.01. Therefore, the effect of B125 was significantly better than that of BBR in both light and dark cycles. As shown in the total heat curve on the left side of FIG. 2C, the heat production (Kcal/kg/h) of mice in the HFD-B125 group was significantly higher than that of the HFD-Saline and HFD-BBR groups. The light cycle and dark cycle statistics on the right side showed that: during the light cycle, the heat production of the three groups of mice was 583.32 mL/kg/h, 731.09 mL/kg/h, and 854.28 mL/kg/h, respectively. The statistical results showed that HFD-B125 vs HFD-Saline: p<0.001, HFD-BBR vs HFD-Saline: p<0.01, HFD-B125 vs HFD-BBR: p<0.01. During the dark cycle, the carbon dioxide consumption of the three groups of mice was 619.86 mL/kg/h, 768.65 mL/kg/h, and 855.04 mL/kg/h, respectively. The statistical results showed that HFD-B125 vs HFD-Saline: p<0.001, HFD-BBR vs HFD-Saline: p<0.05, HFD-B125 vs HFD-BBR: p<0.05. Therefore, the effect of B125 was significantly better than that of BBR in both light and dark cycles. The above results demonstrated that the berberine derivative NBD-125 could significantly enhance the metabolism and heat production of HFD-induced obesity mice, thereby inhibiting weight gain, with an effect significantly superior to that of berberine.

### 3. Influence of B125 on Adipose Production in HFD-Induced C57BL/6 Obesity Mice

After the metabolic cage experiment, the mice were sacrificed, and their subcutaneous WAT, visceral white adipose, and BAT of each mouse in the three HFD groups (HFD-Saline, HFD-BBR, and HFD-B125) were dissected and collected, weighed, and the values were recorded. Then, various adipose/total body weights were used to calculate the average values of total WAT, subcutaneous white adipose, visceral white adipose, and brown adipose contents of mice in each group (n=5) and plotted. The experimental results are shown in FIG. 1B, FIG. 1C, FIG. 1D, and FIG. 1E. BBR and B125 could reduce the white adipose content in mice from 13.63% to 8.69% and 6.88%, respectively, reduce the subcutaneous white adipose from 6.58% to 3.91% and 3.27%, respectively, and reduce the visceral white adipose from 6.78% to 4.78% and 3.61%, respectively. Both could significantly reduce the weight of white adipose in the HFD-induced C57BL/6 obesity mice, while the weight of brown adipose remained unchanged; and B125 had a more significant inhibitory effect on visceral white adipose than BBR (p<0.05).

At the same time, the collected WAT and BAT were subjected to H&E staining, and the number and proportion of white and brown adipocytes of different sizes were counted using software. The results showed that in the WAT of mice injected intraperitoneally with 10 μM/kg B125 (HFD-B125, injected intraperitoneally once every 2 days for 60 days), the number of cells with an area of (2000-5000) μm² was significantly reduced (from 25.89% to 4.72%, p<0.05), while the number of cells with an area of (500-1000) μm² was significantly increased (from 13.25% to 32.53%, p<0.05) (FIG. 1F, FIG. 1G), indicating that B125 could significantly inhibit the enlargement of white adipocytes and exert the effect of inhibiting white adipose production. The effect of B125 was significantly different from that of BBR (p<0.05). Similarly, as shown in FIG. 1F and FIG. 1H, in the BAT of mice injected intraperitoneally with 10 μM/kg B125 (HFD-B125, injected intraperitoneally once every 2 days for 60 days), the number of cells with an area of (200-500) μm² was significantly reduced (from 24.36% to 11.83%, p<0.001), while the number of cells with an area of (10-50) μm² was significantly increased (from 39.38% to 50.65%, p<0.001). In the BAT of mice injected intraperitoneally with 10 μM/kg BBR (HFD-B125, injected intraperitoneally once every 2 days for 60 days), the number of cells with an area of (200-500) μm² was significantly reduced (from 24.36% to 13.79%, p<0.005), and the number of cells with an area of (10-50) μm² was significantly increased (from 39.38% to 47.45%, p<0.01). Combined with the results of FIG. 1E, the comprehensive analysis showed that brown adipocytes had enhanced function and greater potential.

### II. In Vitro Cell Model: Influence of B125 on Preadipocyte Differentiation

### 1. Cultivation and Induced Differentiation of Preadipocytes 3T3-L1 (Shanghai Cell Bank, Chinese Academy of Sciences (Catalog No.: GNM25))

(1) 3T3-L1 preadipocytes were cultured in normal medium at a 5% CO₂, 37° C., and 95% humidity. The inoculation density was about 50% to 60%. When the cell density reached about 90%, they were passaged (for about 2 days to 3 days) to prevent overcrowding of cells from affecting differentiation potential.
(2) The cells were inoculated at 70% of the plating density into a culture dish and cultured in normal medium for 2 days, the medium was changed 1 time every 2 days until the cells were fully adhered, and cell culture was continued for two days to allow the cells to fully undergo contact inhibition.
(3) An induction medium was replaced, which was counted as day 0 at this time, and induced differentiation was conducted for 2 days.
(4) The culture was continued with a maturation medium for 4 days, the medium was changed every 2 days.
(5) A normal medium was changed to continue the culture for 2 days. At this point, it had been 8 days since the start of induced differentiation. Large lipid droplets could be seen under an optical microscope, the cells were continued to culture for 10 days or 12 days to complete differentiation until maturity.
normal medium: 90% low-glucose DMEM+10% calf serum NCS;
induction medium: 90% high-glucose DMEM+10% fetal bovine serum FBS+850 nM Insulin+0.5 mM IMBX+1 μM Dexamethasone+1 nM T3;
maturation medium: 90% high-glucose DMEM+10% fetal bovine serum FBS+850 nM Insulin+1 nM T3.
The cells before induction were represented by Un, indicating “uninduced”, and the time point of induction medium was 0 day or 0 h.

### 2. Influence of B125 on Mitotic Cloning in Early Differentiation of Preadipocytes 3T3-L1 Detected by Flow Cytometry

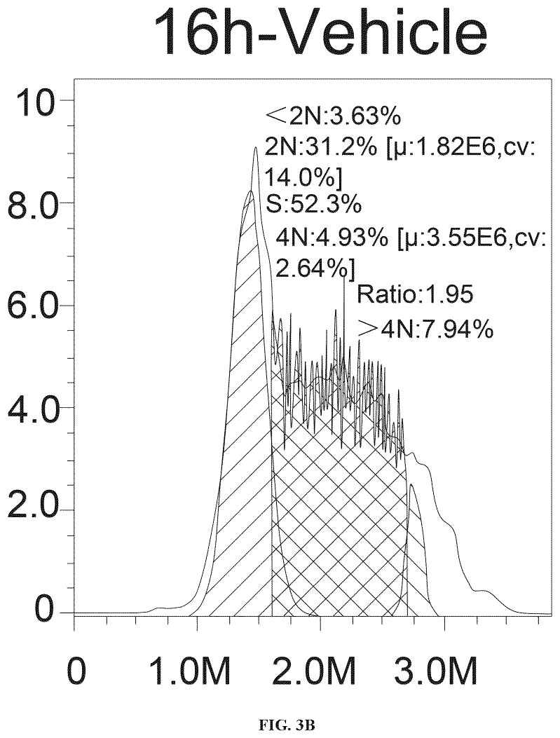

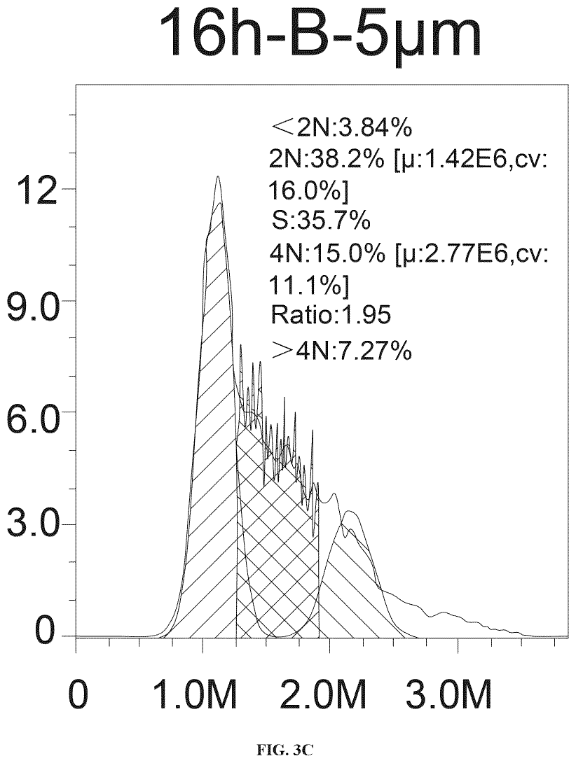

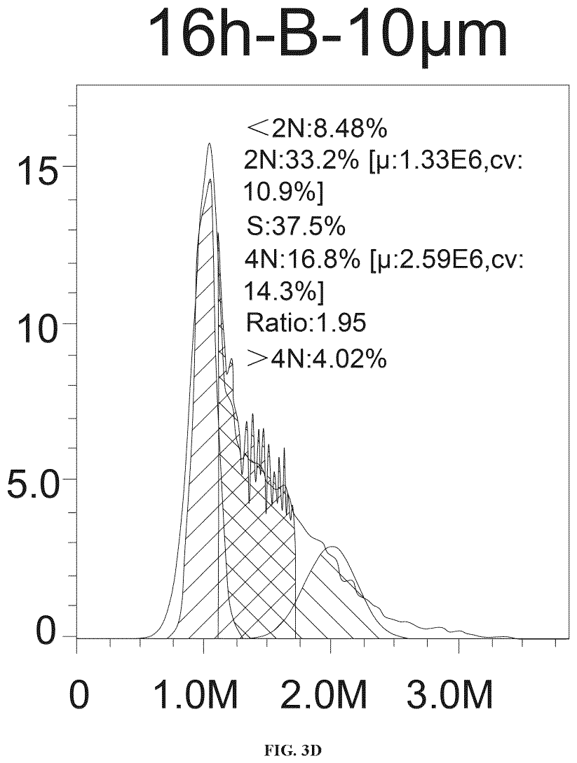

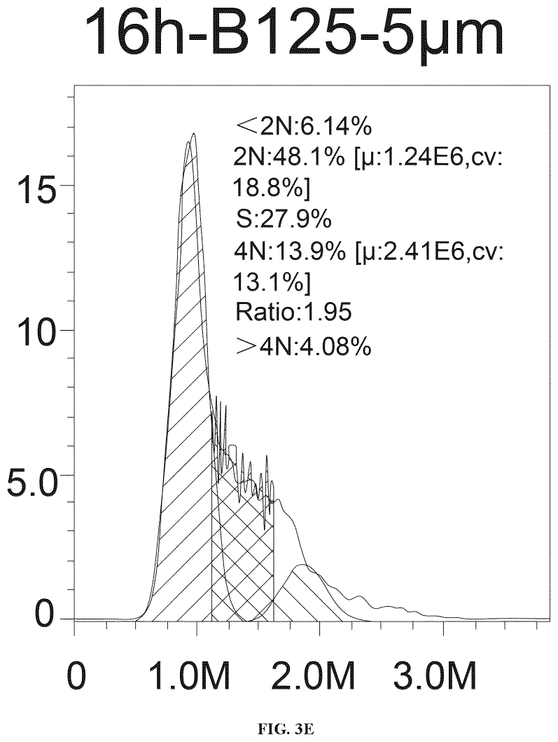

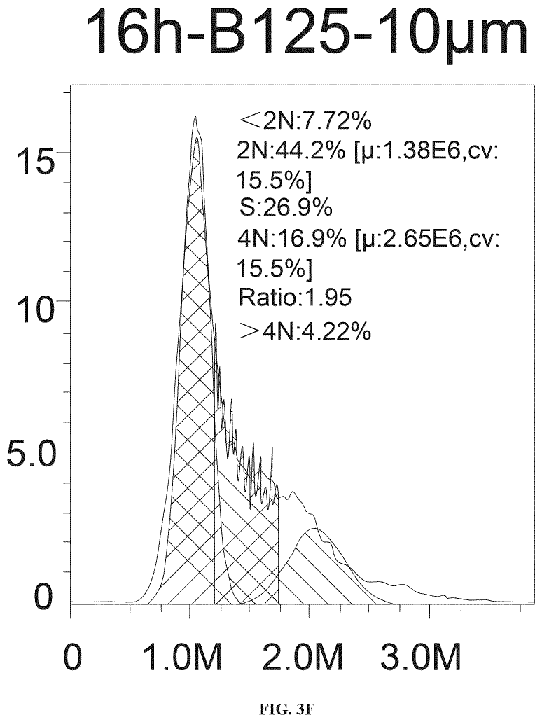

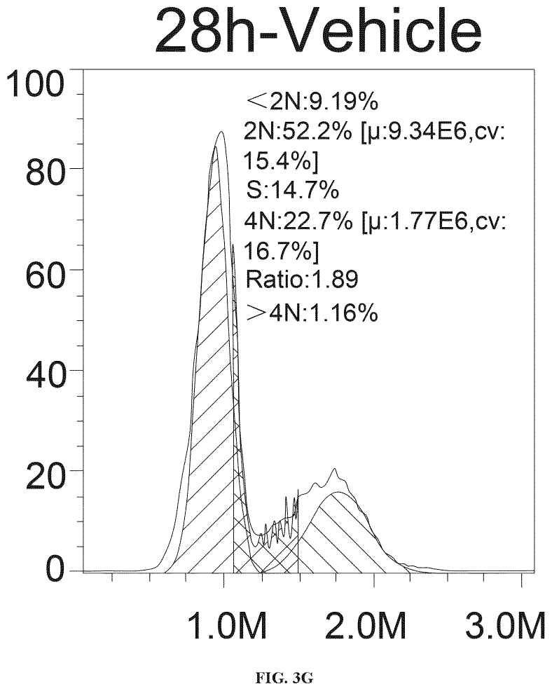

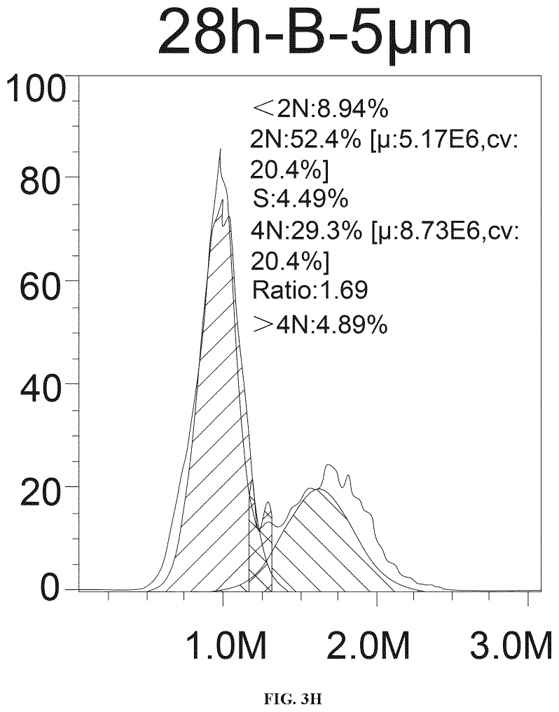

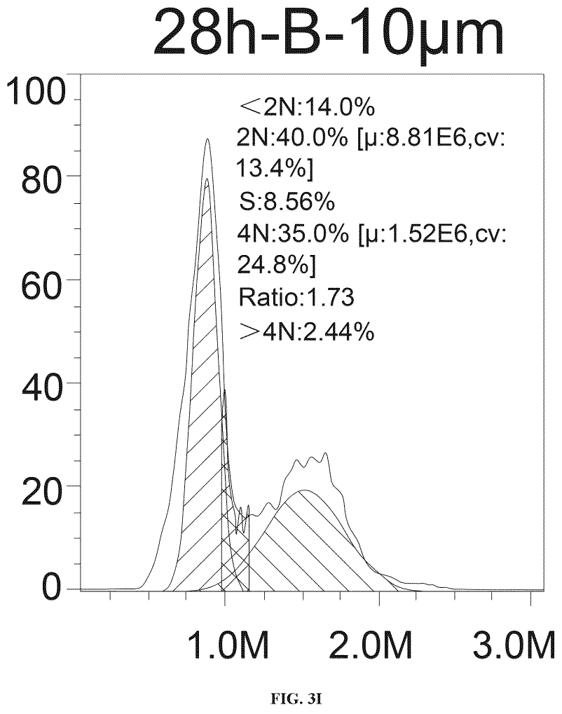

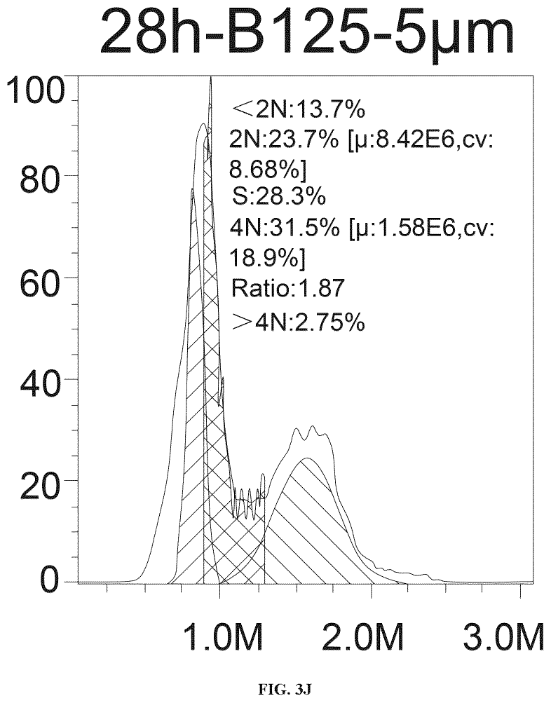

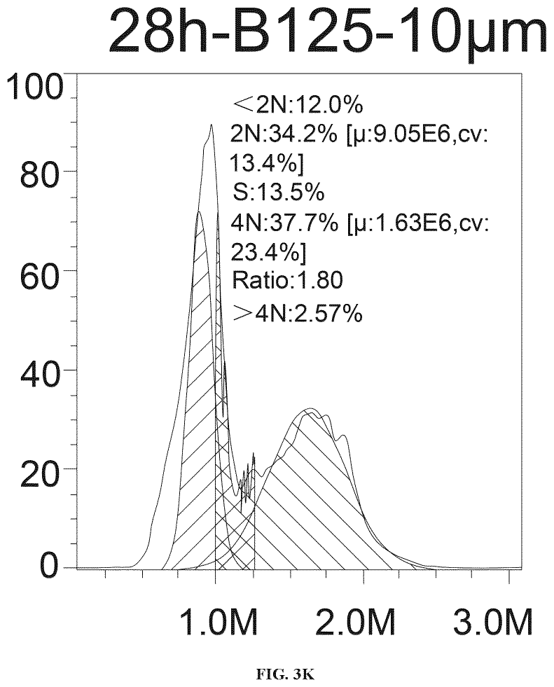

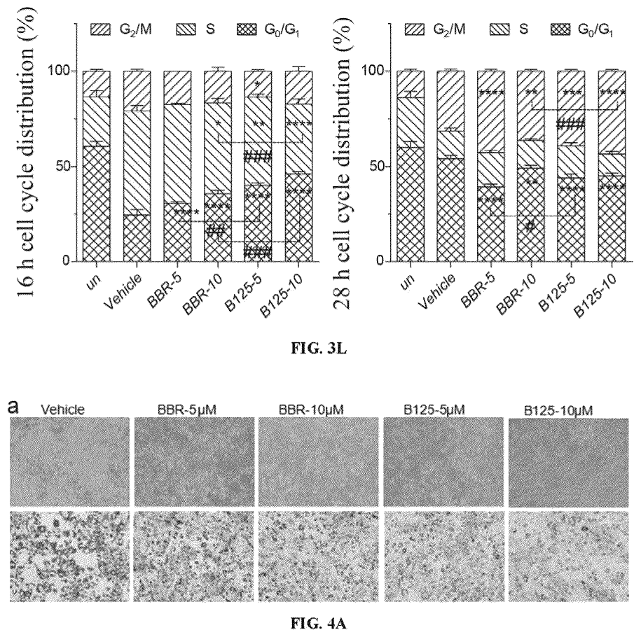

At 0 h (Un) of induced differentiation in preadipocytes 3T3-L1, 0 μM (Vehicle, i.e., DMSO), 5 μM, and 10 μM B125 were added for incubation, and the cells were collected at 16 h and 28 h, respectively. Accordingly, 5 μM and 10 μM BBR were set as positive controls. Cells were fixated with pre-cooled 70% ethanol overnight; the cells were centrifuged at 1,500 rpm, 4° C. for 5 min, and a supernatant was discarded; the cells were resuspended with PBS; 10 mg/mL RNase A was added and incubated for 30 min, centrifuged at 1,500 rpm at 4° C. for 5 min, supernatant was discarded, and the cells were resuspended with 300 μL PBS and placed on ice; 2 μL PI (2 mg/mL) was added for staining for 5 min before loading on machine. The cell cycle was measured by flow cytometry and analyzed using Flow J software, and the results are shown in FIG. 3A-FIG. 3L. After 16 h of treatment with different concentrations of B125, the cell cycle distribution in the early mitotic cloning stage of adipocyte differentiation changed (specific data were shown in Table 1). Combining Table 1 and FIG. 3A-FIG. 3L, after 16 h of treatment with BBR and B125, both could cause an increase in G0/G1 phase cells and a slowdown in mitotic cloning, and the effect of B125 was significantly better than that of BBR; after 28 h of treatment with BBR and B125, both could cause an increase in G2/M phase cells and a slowdown in mitotic cloning, and the effect of B125 was significantly better than that of BBR. Therefore, B125 could significantly block the mitotic cloning of 3T3-L1 in the early stage of differentiation, thereby inhibiting the entire process of adipogenic differentiation, showing an effect significantly better than that of BBR (FIG. 3A-FIG. 3L).

TABLE 1
Cell cycle distribution of preadipocytes at early mitotic cloning
stage of differentiation after 16 h and 28 h of B125 treatment

| Group | G0/G1 (%) | S (%) | G2/M (%) |
|---|---|---|---|
| 0 h-Un | 59.86 | 18.59 | 21.54 |
| 16 h-Vehicle | 35.28 | 59.14 | 5.58 |
| 16 h-BBR-5 μM | 37.94 | 42.86 | 19.2 |
| 16 h-BBR-10 μM | 42.97 | 40.16 | 16.87 |
| 16 h-B125-5 μM | 53.5 | 31 | 15.46 |
| 16 h-B125-10 μM | 50.22 | 30.57 | 19.2 |
| 28 h-Vehicle | 58.26 | 16.41 | 25.33 |
| 28 h-BBR-5 μM | 60.8 | 5.21 | 33.99 |
| 28 h-BBR-10 μM | 47.87 | 10.24 | 41.89 |
| 28 h-B125-5 μM | 28.38 | 33.89 | 37.82 |
| 28 h-B125-10 μM | 40.05 | 15.81 | 44.15 |

### 3. Influence of B125 on Lipid Accumulation in Preadipocytes 3T3-L1 at the Middle and Late Stages of Differentiation

### 3.1 Oil Red Staining and Light Microscopy Observation

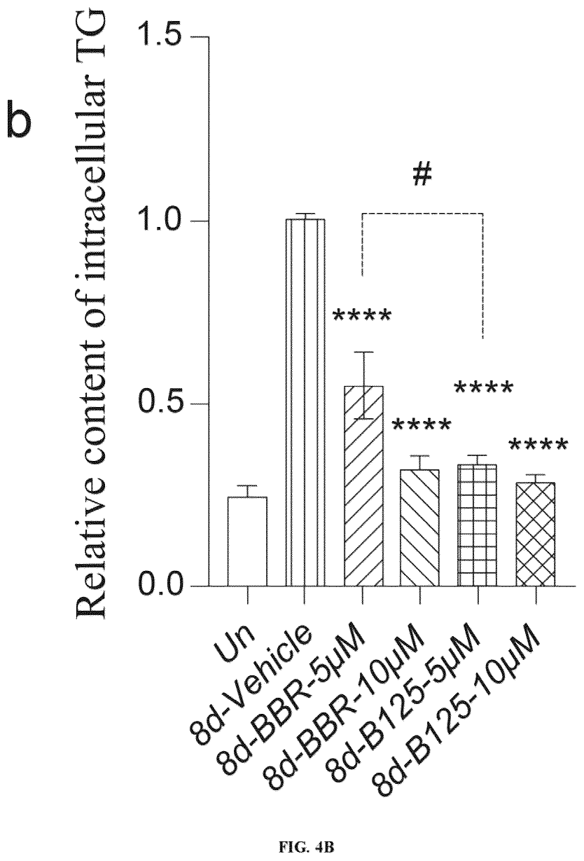

(1) Preparation of oil red stock solution: 150 mg of oil red O powder was added to 50 mL of 99% isopropanol solution and shaken well for later use.
(2) Preparation of oil red working solution: the oil red stock solution was diluted with double distilled water in a ratio of 3:2, mixed well, and filtered with filter paper for later use.
(3) Fixation of cells: cells were taken out from the incubator, the medium of differentiated mature adipocytes was removed, and the cells were washed once with PBS, and fixated with 0.5% glutaraldehyde at room temperature for 1 h.
(4) Washing: after absorbing the glutaraldehyde, the cells were washed again with PBS and dried, and 60% isopropanol was added and allowed to stand at room temperature for 5 min.
(5) Staining: the isopropanol was discarded, an appropriate amount of the previously filtered oil red working solution was pipetted into a sample well, and the culture dish was placed on a shaker and gently shaken for 10 min to evenly distribute the oil red throughout the dish to ensure that the lipid droplets were colored.
(6) Photographing: the oil red staining solution in the culture dish was discarded, the cells were washed several times with ddH₂O, and finally PBS was added. The lipid droplet staining was directly photographed using a phase contrast microscope, and the results are shown in FIG. 4A.

### 3.2 Determination of Triglyceride (TG) Content

At 0 h of induced differentiation in preadipocytes 3T3-L1 (labeled as Un before induced differentiation), 0 μM, 5 μM, and 10 μM B125 were added and incubated for 48 h; the induced differentiation was continued until day 8, and cells in the control group, B125 group, and positive BBR group were collected. The cells were lysed on ice for 15 min using RIPA lysis buffer, sonicated for 5 s/time three times, centrifuged at 12,000 rpm at 4° C. for 20 min, and a supernatant was collected; the protein concentration in the supernatant was determined, and the TG content was determined using an automatic biochemical analyzer according to the instructions. The results are shown in FIG. 4B. 5 μM and 10 μM BBR significantly inhibited lipid accumulation in 3T3-L1 preadipocyte differentiation (inhibition rates were 45.38% and 68.48%, respectively, p<0.001 and p<0.001); 5 μM and 10 μM B125 significantly inhibited lipid accumulation in 3T3-L1 preadipocyte differentiation (inhibition rates were 66.92% and 71.55%, respectively, p<0.001 and p<0.001). This indicated that at a concentration of 5 μM, the inhibitory effect of B125 was significantly better than that of BBR (p<0.05).

Although the above examples have been thoroughly described the present disclosure, they are only some but not all examples of the present disclosure, and other examples may be obtained without inventive steps according to the present examples, all of which fall within the scope of protection of the present disclosure.

What is claimed is:

1. A method for preventing and/or treating obesity, comprising administering a berberine derivative NBD-125 to a subject in need thereof.

2. The method according to claim 1, wherein the obesity comprises high fat diet (HFD)-induced obesity.

3. A method for inhibiting white adipose production, comprising administering a berberine derivative NBD-125 to a subject in need thereof.

4. The method according to claim 3, wherein the inhibiting white adipose production comprises inhibiting white adipose gain and/or inhibiting white adipocyte enlargement.

5. A method for inhibiting differentiation of a preadipocyte into an adipocyte and/or lipid accumulation, comprising administering a berberine derivative NBD-125 to a subject in need thereof.

6. The method according to claim 5, wherein the inhibiting differentiation of the preadipocyte into the adipocyte comprises inhibiting mitotic cloning of the preadipocyte at an early stage of the differentiation.

7. The method according to claim 1, wherein the berberine derivative NBD-125 has a structure shown in formula I. [chemical structure]
